(19) United States
(12) Patent Application Publication
(10) Pub. No.: US 2026/0076756 A1
(43) Pub. Date: Mar. 19, 2026

(54) AUTOMATED INSTRUMENT SEPARATION SYSTEM AND METHOD

(71) Applicant: Aesculap AG, Tuttlingen (DE)

(72) Inventors: Arne Jansen-Troy, Stockach (DE); André-Manuel Eberhardt-Fonseca, Stuttgart (DE); Allan Maas, Konstanz (DE); Tobias Koller, Tuttlingen (DE)

(21) Appl. No.: 19/331,145

(22) Filed: Sep. 17, 2025

(30) Foreign Application Priority Data
Sep. 19, 2024 (DE) 10 2024 126 919.7

(51) Int. Cl. A61B 34/30; A61B 17/00; A61B 34/20

(52) U.S. Cl. CPC A61B 34/30; A61B 17/00; A61B 34/20; A61B 2017/0042; A61B 2017/00544; A61B 2017/00876; A61B 2034/2055; A61B 2034/301

(57) ABSTRACT

A gripping system for gripping and positioning medical instruments includes a gripping device, an adapter device and a holding device. The gripping device includes an electromagnet configured to receive magnetizable instruments from an instrument container. The adapter device attaches the gripping device to a control system, which may be a robotic system. The holding device attaches the electromagnet to the adapter device. The holding device includes a receptacle, which may be a cone, for receiving the electromagnet. The holding device is arranged such that the electromagnet is movable between a first position, in which the electromagnet is received in the receptacle, and a second position, in which the electromagnet is extended out of the receptacle.

### CROSS-REFERENCE TO RELATED APPLICATION

This application claims priority under 35 U.S.C. § 119 to German Application No. 10 2024 126 919.7, filed on Sep. 19, 2024, the content of which is incorporated by reference herein in its entirety.

### FIELD

The present disclosure relates to a gripping system for gripping and positioning medical instruments and a method for removing surgical instruments from an instrument container.

### BACKGROUND

Various methods of handling and processing surgical instruments within medical device reprocessing units (AEMP) are known in the prior art. However, despite considerable advances in automation technology and robotics, the processes involved in cleaning, sterilizing, and preparing these instruments for reuse continue to present challenges. The existing problems include, in particular, the manual handling of sterile containers and sieve baskets, which is not only time-consuming but also involves a considerable risk of physical strain and injury to personnel. Lifting and carrying heavy loads and handling instruments with precision pose an ergonomic challenge and can lead to long-term health problems. In addition, there is the difficulty of ensuring efficient and safe interaction between the gripping system and the objects to be handled, especially when it comes to separating and gripping instruments from chaotic conditions. Such a chaotic state arises, for example, when instruments are placed in a sterile container as bulk goods, i.e., randomly poured and not deliberately arranged. The need to increase operational safety while simplifying maintenance and component replacement poses a further challenge.

### SUMMARY

It is therefore one of the tasks underlying the present disclosure to overcome the disadvantages of gripping devices for gripping and positioning medical instruments in the prior art. In particular, one of the tasks underlying the present disclosure is to provide gripping devices and corresponding methods that allow improved handling and processing of surgical instruments, in particular to increase efficiency and safety in the AEMP and to minimize the risk to personnel.

Preferred embodiments can be taken from the following description.

The present disclosure offers the particular advantage of designing the sorting process for medical instruments from an instrument container, such as a sieve basket, in such a way that instruments stacked on top of each other can be grasped separately from each other. In addition, the gripping system according to the present disclosure allows the instruments to be placed at the desired location, thereby avoiding unnecessary process steps or hindering subsequent process steps.

The method according to the present disclosure allows chaotic arrangements of surgical instruments, especially unclean ones, to be removed randomly from the sieve basket and then stored in a defined manner. The instrument container, e.g., the tray basket or cleaning basket, can then be fed to the cleaning and disinfection devices (RDGs) for further processing steps, e.g., by means of conveyor belts, automated guided vehicles (AGVs), shuttle systems, etc.

The embodiments, features, and combinations of features as described herein in connection with the present disclosure, as well as any combination of features as mentioned and described in connection with the embodiments, are considered to be disclosed herein, or at least to be derivable by a person skilled in the art.

The tasks described above are solved according to the present disclosure by a gripping system for gripping and positioning medical instruments, comprising:
a) a gripping device comprising an electromagnet and designed and provided for receiving magnetizable instruments from an instrument container by means of the electromagnet;
b) an adapter device which is designed and provided for attaching the gripping device to a control system, preferably to a robotic system;
c) a holding device, wherein the electromagnet is attached to the adapter device by means of the holding device;
d) wherein the holding device comprises a receptacle, in particular a cone, for receiving the electromagnet;
and
wherein the holding device is designed such that the electromagnet can be moved between a first position, in which the electromagnet is received in the receptacle, and a second position, in which the electromagnet is extended out of the receptacle.

The tasks described above are also solved by a method according to the present disclosure for removing surgical instruments from an instrument container, in particular a sieve basket, and in particular for separating and handling the surgical instruments. Such a method according to the present disclosure comprises at least one step of providing a gripping system and
optionally a step a) of activating the electromagnet;
a step b) of moving the electromagnet from a first position, in which the electromagnet is received in the receptacle, to a second position, in which the electromagnet is extended out of the receptacle;
a step c) of receiving a magnetizable instrument by means of the electromagnet in the second position;
a step d) of moving the electromagnet from the second position, in which the electromagnet is extended out of the receptacle, to the first position, in which the electromagnet is received in the receptacle;
optionally a step e) of deactivating the electromagnet;
wherein the gripping system is preferably a gripping system according to the present disclosure.

In connection with the method according to the present disclosure, it should also be noted that the steps indicated do not necessarily have to be carried out in the order indicated. The steps indicated can be carried out in any other suitable order or even simultaneously.

However, the order specified above may be advantageous for certain embodiments of the method according to the present disclosure. At the same time, for example, step a) of activating the electromagnet may be carried out before or after step b) of the electromagnet process from a first position, in which the electromagnet is received in the receptacle, to a second position, in which the electromagnet is extended out of the receptacle, so that in step c) it is then possible to receive a magnetizable instrument by means of the electromagnet in the second position. Step b) of moving the electromagnet from a first position, in which the electromagnet is received in the receptacle, to a second position, in which the electromagnet is extended out of the receptacle, can also be performed before step c) and then step d). This means that the electromagnet is extended, and then an instrument is gripped so that the electromagnet with the gripped instrument can then be returned to the first position in the holder. However, it is also conceivable, for example, that the gripping system is already in a second position in which the electromagnet is extended out of the holder, and a magnetizable instrument can be picked up immediately in step b) by means of the electromagnet, or, optionally, in step c), the electromagnet is moved from the second position, in which the electromagnet has been extended out of the receptacle, to the first position, in which the electromagnet is received in the receptacle. The skilled person will therefore immediately recognize that different sequences of the steps of the method according to the present disclosure are advantageous according to the present disclosure.

This is particularly advantageous because it significantly increases the flexibility and efficiency of the gripping system.

Step a) of activating the electromagnet is particularly necessary if the electromagnet has been deactivated beforehand. In the simplest case, the electromagnet is activated, an instrument is gripped, and the electromagnet is deactivated again to release the instrument, so that it is expedient to reactivate the electromagnet for each gripping cycle. However, it is also within the scope of the present disclosure to leave the electromagnet permanently activated and to achieve the release of the instruments by means of a stripping action according to the present disclosure while the electromagnet is activated. In such a case, it is not necessary to reactivate the electromagnet.

The ability to move the electromagnet between two positions allows the system to be used optimally for both precise gripping and safe positioning of medical instruments. This helps to automate the handling of instruments and reduce the workload of personnel, while at the same time improving the precision and reliability of the processes within the AEMP. In addition, the use of an electromagnet minimizes the risk of contamination by reducing direct contact between the instruments and the operating personnel. Integration into a robotic system also expands the possible applications of the gripping system, allowing it to be adapted to different working environments and tasks. Instrument containers, especially in the form of sterile goods containers, such as a sieve basket or similar, can be handled with the gripping system according to the present disclosure, and instruments can be removed individually from the instrument container.

In the method according to the present disclosure or in the gripping system according to the present disclosure, the electromagnet is located in the first position within the holder, specifically a cone. This configuration is particularly advantageous for positioning the magnetizable instruments in a defined manner. In particular, the electromagnet can be positioned in the receptacle and stabilized against unwanted movements in such a way that the unintentional release or falling of the gripped instruments is prevented as far as possible, even when the robot arm of the robotic system is moving. According to the present disclosure, it is preferred that in the first position, the receiving surface or magnetic surface of the electromagnet is approximately flush with the edges of the holder or cone.

According to the present disclosure, it is provided that the electromagnet can be switched on and off in order to switch between a state in which the electromagnetic field of the electromagnet is active and an instrument can be gripped and held, and a state in which the electromagnet is switched off and the electromagnetic field of the electromagnet is not active. However, the skilled person will immediately recognize that in any state in which an instrument is to be held or held as intended, the electromagnet will be active, and in any state in which an instrument is not to be held or is to be removed, it may be switched off. It should also be understood that in the stripping described below as according to the present disclosure, the electromagnet is preferably switched off, but this is not necessarily essential.

According to the present disclosure, the electromagnet is extended out of the receptacle in the second position. This makes it particularly easy to place the electromagnet over an instrument, especially when switched on, i.e., with an active electromagnetic field. This facilitates contact between the electromagnet and the instrument and, if necessary, also increases the precision with which the instrument can be activated and picked up by the electromagnet. This is particularly the case when the instrument is stored in a disorderly manner, for example inside a sterile goods container or on a surgical tray.

It should be understood that the ability of the system according to the present disclosure, and in particular of the method according to the present disclosure, to switch between the first and second positions increases the versatility of the gripping system and contributes to the optimization of sterile processes.

It will also be recognized by a person skilled in the art that a feature, embodiment, effect, or advantage as described here in connection with the inventive device and/or the inventive reverse osmosis system may also be a feature, embodiment, effect, or advantage of the inventive method, and vice versa.

In connection with the present disclosure, it should also be understood that the gripping system according to the present disclosure can be attached to a robotic system, preferably an articulated arm robot, via the adapter device.

In a further preferred embodiment of the method and/or gripping system according to the present disclosure, the gripping system according to the present disclosure comprises the robotic system, preferably an articulated arm robot.

In a further preferred embodiment of the method and/or gripping system according to the present disclosure, the robotic system is movable in at least three, preferably six, degrees of freedom.

In the present description, unless the context otherwise requires, the word “comprising” and variations thereof are to be understood as implying the inclusion of a specified element, an integer or a step or a group of elements, integers or steps, but not the exclusion of another element, another whole number or another step or another group of elements, whole numbers or steps, although in some embodiments such other elements, whole numbers or steps or groups of elements, whole numbers or steps may be excluded, i.e., the subject matter consists in the inclusion of a specified element, a whole number or a step or a group of elements, whole numbers or steps.

The terms “a” and “an” and “the” and similar references used in the context of the description of the present disclosure are to be interpreted as covering both the singular and the plural, unless otherwise specified herein or clearly contradicted by the context.

Within this application, terms such as “side,” “lateral,” “rear,” “front,” “top,” “bottom,” “bottom,” “opposite,” “inside,” “outside,” or the like, which describe the position of a first object relative to another object, preferably refer to the relative position of a respective part or object in relation to its position when fully assembled for its intended use.

In a preferred embodiment of the method and/or device according to the present disclosure, the holding device is designed such that the electromagnet can be moved from the first position and/or the second position to a third position in which the electromagnet is retracted into the receptacle.

The method according to the present disclosure preferably comprises a step c) of retracting the electromagnet from the first position and/or the second position into a third position in which the electromagnet is retracted into the receptacle.

This advantageously strips the instrument from the receptacle or the cone.

More precisely, by moving the electromagnet into the third position, i.e., retracting it into the receptacle, the grasped instrument advantageously comes into contact with the receptacle and preferably in particular with an edge of the cone. Since the electromagnet no longer exerts any magnetic attraction, the instruments are stripped off by the movement and physical contact with the receptacle and preferably in particular with an edge of the cone.

This is particularly advantageous as it provides an efficient and elegant solution to the problem of residual magnetization. The further retraction of the electromagnet into the cone and the stripping of the instruments at the edge of the cone eliminates the need for an additional demagnetization mechanism. This simplifies the design of the gripping system, reduces the number of moving parts and thus also the maintenance requirements and potential sources of error. In addition, this method enables instruments to be released quickly and reliably, which speeds up and optimizes the entire process of handling and reprocessing medical instruments. The avoidance of additional stripping mechanisms such as wipers also contributes to cost efficiency, reduces the complexity of the system, and improves hygiene management with regard to the prevention of carryover or contamination.

The expression or term “retracted into the receptacle” as used herein preferably means a positioning of the electromagnet and, if applicable, the elements connected thereto, such as a positioning means, in which the electromagnet and, if applicable, the elements connected thereto are moved from an exposed or extended position into a retracted or withdrawn position within the receptacle. In particular, a magnetic surface and/or receiving surface of the electromagnet is retracted relative to the receptacle and, in particular, the receptacle edge or the cone and, in particular, the cone edge, with reference to an instrument to be gripped or gripped.

In a further preferred embodiment of the method and/or gripping system according to the present disclosure, the holding device comprises a steering means which is designed and arranged to move the electromagnet substantially vertically, preferably substantially perpendicularly, relative to a working surface, wherein the steering means preferably comprises at least one flexibly bendable section.

In this context, the term “flexible” as used herein is preferably understood as a property of a material or component that can be flexibly deformed under load without losing its structural integrity. This means that the material or component is capable of adapting to different shapes and positions without breaking or becoming permanently deformed. Examples of bendable materials are ropes, in particular steel ropes, and bendable rods, which may contain joints or flexible sections.

The preferred steering means is a cable, preferably a steel cable, and/or a rod. In this context, it should be understood that the positioning means can be attached to the instruments, which may also be in an inclined position due to the chaotic arrangement in the screen basket, and that the positioning means is preferably designed to be tiltable. According to the present disclosure, the tiltable design is preferably achieved by the steering means comprising at least one flexible section. For example, a cable can be used as the steering means. Alternatively, other steering means can also be used. For example, the steering means can be designed as a rod that is flexible at least in sections. The rod, which is flexible at least in sections, then comprises, for example, a joint or another flexible section, such as a rope or thread section, as the flexible section.

This is particularly advantageous because the steering means enables precise and controlled movement of the electromagnet. The vertical, preferably perpendicular alignment to the work surface ensures that the instruments can initially be raised or lowered directly and without lateral displacement. This minimizes the risk of collisions or misplacement and contributes to increased accuracy in the positioning of the instruments. The use of a cable, preferably a steel cable, or a rod as a steering means also provides a robust and durable solution that is necessary for the precise and repeatable movements within the gripping system. In addition, the “ ” design allows easy integration into existing robotic systems and contributes to the modularity and scalability of the overall system.

In addition or as an alternative, the use of a steering means, in particular in the form of a cable, preferably a steel cable, also allows the electromagnet to be mounted so that it can swing freely in an extended second position.

In this case, it is particularly advantageous for the electromagnet to be freely suspended in the second position, in which the electromagnet is extended from the holder, on the steering means, in particular a cable, preferably a steel cable. This can be particularly advantageous when lowering the electromagnet onto the instrument to be gripped, since the free-swinging mounting of the electromagnet, in particular on a cable, preferably a steel cable, in the second position allows the gripping system to adapt more easily to different object geometries. The free movement of the electromagnet allows it to align itself independently in the optimal position above the instrument to be gripped, which improves the precision of the gripping process and minimizes the risk of misalignment or damage to the instruments. In addition, the oscillating mounting can help to distribute the forces acting on the instruments during gripping, thus ensuring gentler handling.

In embodiments in which the steering means is a cable, preferably a steel cable, the electromagnet can advantageously be mounted at a first end of the cable, preferably the steel cable, which is attached to the holding device at a second end. In particular, the holding device may comprise a guide, e.g. a precision tube, which guides the cable, preferably steel cable, at least in sections. The cable, preferably steel cable, may advantageously be mounted at least in sections within the precision tube.

Furthermore, the holding device can advantageously comprise deflections, for example in the form of sleeves. This allows the cable, preferably steel cable, to be deflected advantageously within the gripping device. For example, a deflection sleeve can be used to deflect the cable, preferably steel cable, from a horizontal guide by 90° into a vertical position.

The term “working surface” as used herein preferably refers to the bottom of the instrument container on which the medical instruments are placed. This surface is designed to provide stable and secure storage for the instruments while allowing easy access for the gripping system to handle the instruments without risk of contamination or damage.

In a further preferred embodiment of the method and/or gripping system according to the present disclosure, the holding device comprises a drive unit ( ), in particular a pneumatic unit, which is designed and arranged to move the electromagnet and/or the steering means.

In particular, a cable, preferably a steel cable, can also be used as a steering means to raise or lower the electromagnet and thus move precisely between the various positions according to the present disclosure.

In a further preferred embodiment of the method and/or gripping system according to the present disclosure, the holding device comprises a pneumatic unit as a drive unit, which comprises a pneumatic cylinder that is preferably designed and set up to move the electromagnet and/or the steering means by means of at least two different pressure stages of the pneumatic cylinder.

This is particularly advantageous because the use of a pneumatic unit with a pneumatic cylinder that has at least two different pressure stages enables differentiated and precise control of the gripping system. The different pressure stages allow finely tuned movement of the electromagnet and/or the steering means, and thus improved movement between the various positions of the electromagnet according to the present disclosure.

For example, in a first pressure stage, the electromagnet can be moved into the first position, i.e., it can be retracted into the receptacle or the outer cone, in order to stabilize the electromagnet relative to the entire gripping system, and in a second pressure stage, it can be moved further into the outer cone into a third position in order to strip magnetizable instruments after the electromagnet is switched off.

In a further preferred embodiment of the method and/or gripping system according to the present disclosure, the electromagnet comprises a positioning means. Such a positioning means is particularly advantageous for temporarily stabilizing the electromagnet within the receptacle with the aid of the positioning means in the form of a device or mechanism. This makes it possible to hold the electromagnet in a defined position during operation without it wobbling or performing unwanted movements. This is particularly advantageous for ensuring precise handling of the medical instruments while maintaining the flexibility of the system.

It is preferable that the positioning means is designed as a counterpart to the receptacle. For example, the positioning means can be designed as a counterpart to an outer cone. Such a positioning means can serve as an element for guiding the electromagnet in the receptacle and, in particular, when entering the outer cone. This can, for example, help to ensure that the electromagnet is correctly aligned and enters the cone in a stable manner. Furthermore, in a first pressure stage, for example, when the electromagnet enters the holder or the outer cone, the counterpart can help to stabilize the electromagnet. This advantageously prevents unwanted movements and ensures a secure holding position. At the same time, the counterpart can be shaped so that it enlarges the mounting surface for an instrument, especially around a mounting surface of the electromagnet, so that the mounted instrument can be held more securely. Furthermore, particularly in the second pressure stage, the counterpart can assist in stripping off instruments that are still adhering to the electromagnet due to residual magnetization. By further moving the electromagnet into the cone, the instruments are stripped off at the edge of the counterpart.

In a further preferred embodiment of the method and/or gripping system according to the present disclosure, a control unit is also provided which is configured to transmit control commands to the drive unit for moving the electromagnet. Such a control unit can be implemented at various points in the gripping system and, in particular, can also be designed as part of a robotic system.

The control unit is used to transmit control commands to the drive unit for moving the electromagnet, so that the electromagnet can be moved between the various positions according to the present disclosure. Furthermore, the control unit can preferably transmit control commands to the drive unit to switch the electromagnet on or off, so that the electromagnet can switch between a state in which the electromagnetic field of the electromagnet is active so that an instrument can be picked up and held, and a state in which the electromagnet is switched off and the electromagnetic field of the electromagnet is not active.

Also according to the present disclosure, in order to connect a control unit to the holding device and in particular to the electromagnet for control purposes, the adapter device may have corresponding interfaces which are designed and provided for transmitting control commands between the control unit and the holding device and in particular the electromagnet.

In a further preferred embodiment of the method and/or gripping system according to the present disclosure, the control unit is designed to detect instruments in an empty instrument container, preferably by means of image data captured by an optical system. In a preferred embodiment, the gripping system according to the present disclosure comprises an optical system which is designed in particular to detect information about the instrument container and, if applicable, its contents. Such an optical system may comprise optical sensors, in particular a camera. The optical system and/or the control unit may be advantageously designed such that an evaluation of the collected sensor data of the optical system is used to check whether all instruments have been gripped and/or whether instruments are present in the instrument container.

The gripping system and, in particular, the control unit and/or the optical system are preferably trained to detect an empty instrument container, in particular a sieve basket. The control unit can advantageously comprise a neural network and/or be in communication with such a neural network. In particular, such a neural network is advantageously trained to detect an empty instrument container, in particular a sieve basket. Accordingly, the method and/or gripping system according to the present disclosure may comprise that, in a case where instruments are still detected during movement and/or optical scanning in different areas, these are approached by the gripping system in such a way that the electromagnet is located vertically above one of the instruments. These instruments can then be removed individually and one after the other by means of the gripping system according to the present disclosure until they have all been removed. A person skilled in the art will immediately recognize that this method according to the present disclosure represents a departure from the prior art, in which essentially corresponding systems are trained to detect the presence of instruments.

The method according to the present disclosure preferably comprises a step of scanning and/or optical scanning by means of the optical system, wherein the instrument container is scanned and any instruments present are detected, preferably as described above. Furthermore, a step of scanning and/or optical scanning by means of the optical system comprises translational scanning and/or translational optical scanning of the instrument container in a three-dimensional grid.

In a further preferred embodiment of the method and/or gripping system according to the present disclosure, the holding device further comprises at least one compression spring, which is preferably arranged and positioned such that the compression spring is compressed by the dead weight of the electromagnet and, if applicable, further components, in particular the components connected to the electromagnet, in particular a positioning means. Such a compression spring is preferably arranged horizontally in its compression direction, i.e., preferably orthogonally to the main gripping direction of the electromagnet.

For example, when the steering mechanism, particularly in the form of a cable, preferably a steel cable, is relieved, by placing the electromagnet on the bottom of the instrument housing and/or an instrument, this pressure spring can be arranged and set up in such a way that it is compressed by the dead weight of the electromagnet and all other components. When the electromagnet is placed on the ground and the cable, preferably a steel cable, is relieved, the pressure spring then expands and thus guides the cable, preferably a steel cable, within the holding device, and in particular, if present, with to a guide for the cable, preferably a steel cable, e.g., a tube, in particular a precision tube. This prevents the rope, preferably steel rope, from becoming jammed in the holding device, in particular a guide for the rope, preferably steel rope, e.g. a tube or a deflection device if present, due to the release of the load.

In a further preferred embodiment of the method and/or gripping system according to the present disclosure, the holding device has at least one positioning spring ( ) on the electromagnetic gripper ( ), which is arranged and designed to position the electromagnetic gripper in the holder.

In a further preferred embodiment of the method and/or gripping system according to the present disclosure, the holding device is designed such that the electromagnet can be moved, preferably from the third position, into a fourth position in which the electromagnet is drawn into a feed channel of the positioning means.

In particular, a method according to the present disclosure may comprise a step in which the electromagnet, preferably from the third position, is pulled into a fourth position into a feed channel of the positioning means.

In this case, a pneumatic cylinder may be provided and set up for this purpose, which moves the electromagnet and/or the steering means into such a fourth position by means of at least one further pressure stage of the pneumatic cylinder.

In particular, a further spring may be arranged between the electromagnet and, if applicable, the positioning means, which spring is arranged around the steering means, in particular the cable, preferably a steel cable. This further spring preferably has a higher spring constant than the positioning spring. The spring is preferably arranged between the magnet and the upper end of the positioning means, in particular the upper end of the pull-in channel of the positioning means. In a further preferred embodiment, the pull-in channel is partially, preferably completely, closed by a cover at its end intended for instrument reception. This makes it particularly advantageous to strip off instruments, in particular pointed instrument parts, which would otherwise be pulled into the pull-in channel while hanging on the magnet without being stripped off.

Approaching the fourth position in accordance with the present disclosure is particularly advantageous if the stripping of instruments in a third position was not successful. Simultaneously or alternatively, the electromagnet can be moved to the fourth position simultaneously or instead of being moved to the third position. In other words, the gripping system is then set up in such a way that the electromagnet can immediately retract into the feed channel for stripping.

Retracting the electromagnet into the feed channel is particularly advantageous if an instrument with the instrument syringe, i.e., essentially in a vertical alignment with the instrument container bottom, is hanging on the electromagnet and thus does not come into contact with the receptacle when the electromagnet is moved into a third position, i.e., preferably into the receptacle.

In a further preferred embodiment, the gripping system according to the present disclosure comprises a force or strain sensor which is designed to detect unsuccessful stripping of an instrument in the third position. Optionally, unsuccessful stripping can be detected simultaneously or alternatively by optical means, in particular by the optical system.

Alternatively, the fourth position can be performed after a step of retracting the electromagnet from the first position and/or the second position into a third position, in which the electromagnet is retracted into the holder (11), thereby stripping off the instrument. In this case, a corresponding sensor system could optionally be omitted.

If the fourth position is now approached and the pressure increased, the electromagnet moves upward in the positioning means, in particular the retraction channel, thereby compressing the further spring.

A relatively thin but rigid cover may preferably be provided on the underside of the positioning means, which covers the electromagnet downwards and is preferably neither magnetic nor magnetizable itself. The cover prevents the instrument adhering to the e from following the electromagnet into the positioning means or the feed channel. This has the advantage of mechanically separating the electromagnet and the instrument from each other so that the instrument detaches from the electromagnet and the positioning means and is thus deposited.

In a further preferred embodiment of the gripping system according to the present disclosure, the gripping system comprises a protective cell. Such a protective cell can, in particular, increase the system safety of the gripping system according to the present disclosure. Preferably, the entire gripping system is housed in a corresponding protective cell, for example consisting of aluminum profiles and PC plates with corresponding openings for conveyor belts or other interfaces. Furthermore, openings can be provided in the protective cell to prevent accidents and unauthorized access by means of light barriers and other protective mechanisms.

In a further preferred embodiment of the gripping system according to the present disclosure, the gripping system comprises a suction gripper. The suction gripper can be additionally equipped to enable the gripping of trays or non-magnetizable instruments with smooth surfaces.

### BRIEF DESCRIPTION OF THE DRAWINGS

The present disclosure is explained in more detail below with reference to the drawings, from which further features, embodiments, and advantages can be derived. In the embodiments shown in the figures, elements that have similar or identical functions are designated by the same reference numerals. It should be noted that the figures may not be to scale with respect to each other.

The following is shown:

FIG. 1 shows a schematic representation of a gripping system according to a first embodiment of the present disclosure;

FIG. 2 shows a schematic sectional drawing of a gripping system according to the first embodiment of the present disclosure in a second position;

FIG. 3 shows a schematic sectional drawing of a gripping system according to the first embodiment of the present disclosure in a centering position;

FIG. 4 shows a schematic sectional drawing of a gripping system according to the first embodiment of the present disclosure in a third position.

FIGS. 5a and 5b show schematic sectional drawings of a gripping system according to the first embodiment of the present disclosure in a third (FIG. 5a) and in a fourth (FIG. 5b) position.

### DETAILED DESCRIPTION

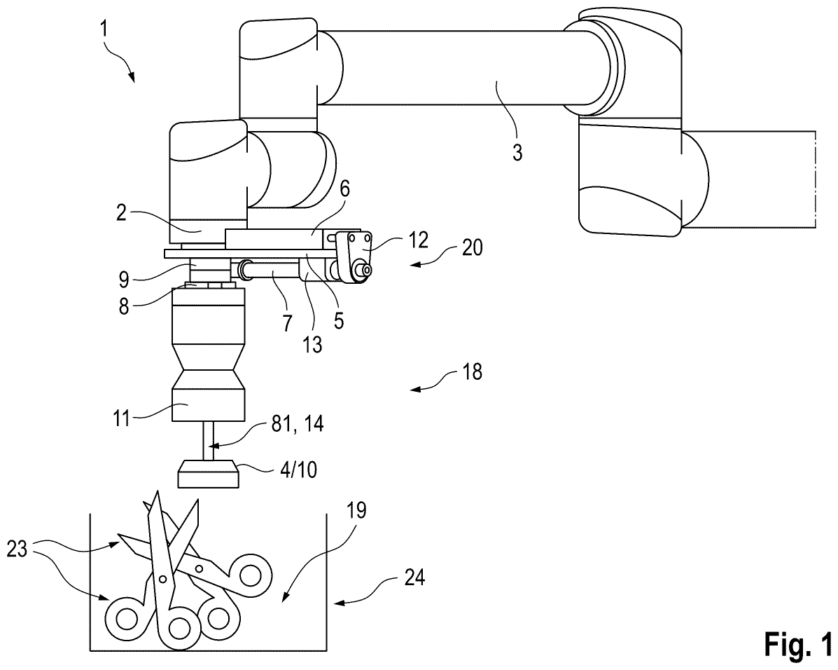

FIG. 1 shows a schematic representation of the gripping system according to a first embodiment of the present disclosure. The gripping system is suitable for gripping and positioning medical instruments 23. FIG. 1 shows how the gripping device 1 is attached to a robotic system 3, in this case a 6 DOF robot arm, by means of an adapter device 2. This has the advantage that the gripping system as a whole can be moved in an automated and controllable manner in at least three degrees of translational freedom, or alternatively in 6 DOF with the aid of an articulated arm robot 3.

The gripping device 1 comprises the electromagnet 4 for picking up magnetizable instruments from an instrument container. For this purpose, the electromagnet is provided in the holding device 18, whereby the electromagnet 4 is also attached to the adapter device 2 and thus to the control system, here a robotic system 3, by means of this holding device 18. The holding device 18 has a holding receptacle 11 in the form of a cone 11, in which the electromagnet 4 can be received.

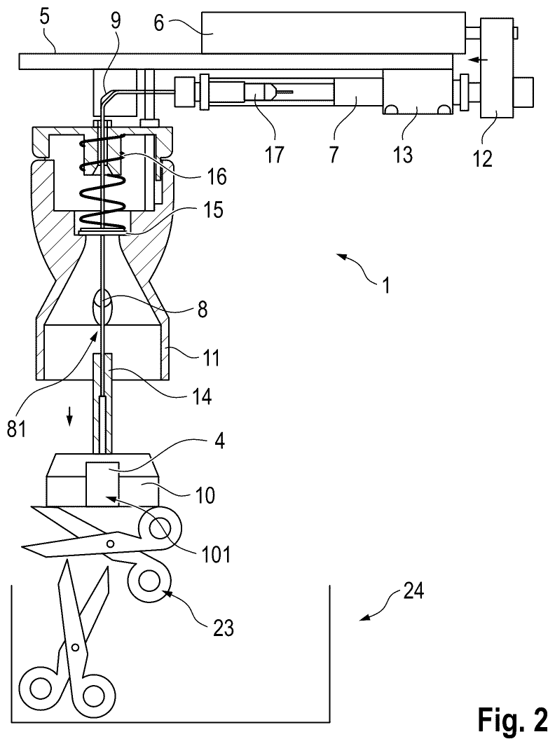

FIG. 1 shows the gripping device in a second position, in which the electromagnet 4 has been moved out of the receptacle 11. According to the present disclosure, the holding device 18 is designed in such a way that the electromagnet 4 can be moved from the second position shown to the first position, in which the electromagnet 4 is received in the receptacle 11. According to the present disclosure, the electromagnet 4 can also be moved from the first position, in which the electromagnet 4 is received in the receptacle 11 (see FIG. 3, in which this first position has not yet been completely reached), to the second position, in which the electromagnet 4 is extended out of the receptacle 11 (FIG. 1 and FIG. 2). In this second position, the electromagnet 4 can pick up instruments 23 from the sieve basket 24 with the required flexibility.

FIG. 1 shows the assembly of the gripping system comprising the robotic system 3, the adapter 2, and the holding device 18 with electromagnet 4. In the position shown, the electromagnet 4 can remove instruments from the instrument container by means of the method according to the present disclosure.

The holding device 18 has both the steel cable 81 and the rod 14 as steering means 8, so that the steering means comprises at least one flexible section. The steering means 8 allow the electromagnet 4 to be moved substantially vertically, preferably substantially perpendicular, to a working surface 19. The cable 81, preferably a steel cable, has a diameter of around 0.5 mm to 1 mm and is connected to a linear actuator with at least one degree of freedom in accordance with the state of the art.

The power supply cable for the electromagnet 4 is preferably laid along the cable 81 and preferably reinforced to prevent kinking. The actuator is connected to the base of the gripper. When lowered, i.e., in the second position, in which the electromagnet 4 is extended from the receptacle 11, the cable 81 or the electromagnet 4 is mounted on the steering means 8, in particular the steel cable 81, so that it can swing freely to enable it to be placed on the instruments 23 without transverse forces and without causing a collision or tilting due to other chaotically arranged instruments 23 or undercuts in the sieve basket 24.

FIG. 2 shows a schematic sectional drawing of the gripping system according to the first embodiment of the present disclosure in a second position, and FIG. 3 shows a schematic sectional drawing of the gripping system according to the first embodiment of the present disclosure in a centering position shortly before the first position is completely reached.

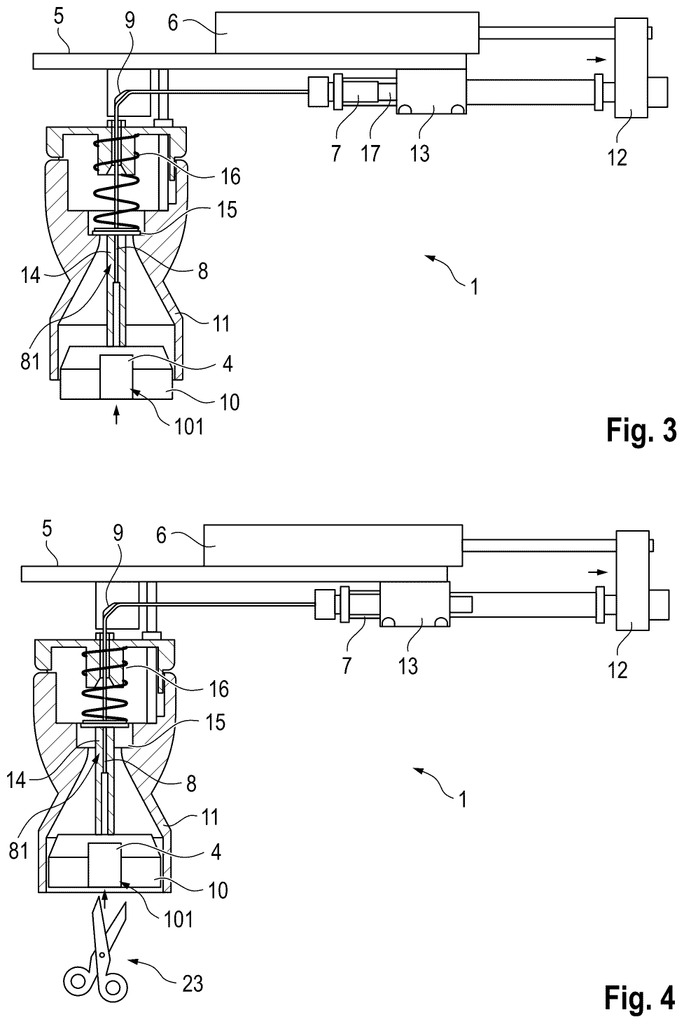

The holding device 18 also allows the electromagnet 4 to be moved from the first position and/or the second position to a third position, which is shown in FIG. 4. This FIG. 4 shows a schematic sectional drawing of the gripping system according to the first embodiment of the present disclosure in a third position, in which the electromagnet 4 is retracted into the receptacle 11. The arrows indicate the direction of movement of the components.

By means of the steering means 8, namely the steel cable 81 and the rod 14, the electromagnet 4 can be moved essentially vertically and perpendicular to the working surface 19. For this purpose, the holding device 18 comprises a drive unit 20 in the form of a pneumatic unit 20. Since the cable 81 is connected at one end to the electromagnet 4 and at its other end to the holding device 18, in particular to the pneumatic unit 20, the electromagnet 4 can be moved by means of the pneumatic unit 20. The double-acting pneumatic cylinder 6 with a stroke of preferably 100 mm is mounted on the upper side of the adapter plate 5. A precision tube 7 is moved in a slide bearing 13 by means of an adapter on the pneumatic cylinder 6 in line with the extension movement of the pneumatic cylinder 6. The cable 81, preferably a steel cable, mounted inside the precision tube 7 is deflected by 90° via an outer sleeve 9. At the end of the cable 81, preferably a steel cable, the electromagnet 4 is mounted with a counterpart 10 to the outer cone 11 as a positioning means 10.

The pneumatic unit 20 acts as the drive unit 20 and comprises the pneumatic cylinder 6, which moves the electromagnet 4 and the cable 81 by means of the various, preferably preset pressure stages of the pneumatic cylinder 6. As shown in FIG. 3, the electromagnet 4 can be retracted into the holder 11 here in the outer cone, with the pneumatic cylinder 6 being operated in the first pressure stage. In order to be able to move the instruments 23 resting on the electromagnet 4 or removed from the sieve basket 23 safely and in a spatially defined manner, i.e. in 6 DOF, the present disclosure proposes that the electromagnet 4 be retracted in the first position, in which the electromagnet 4 is in the receptacle 11, i.e. the outer cone 11 (see FIG. 3). This allows the electromagnet 4 to be stabilized relative to the entire gripping system. Only rotation along the vertical axis of the preferably round electromagnet 4 is still possible in this configuration.

FIG. 4 shows the gripping device 1 according to the present disclosure in a third position, the release position for instruments 23.

Since the magnetizable instruments 12 may retain some residual magnetization after contact with the electromagnetic field of the electromagnet 4, it is proposed to strip them additionally after switching off the electromagnet 4. This may be necessary if, as described at the beginning, residual magnetization is present or the electromagnet 4 is operated in continuous operation.

It should be understood that even if the electromagnet 4 is switched off, especially in the case of light instruments 23, such as tweezers, the residual magnetization may be sufficient to prevent the instruments 23 from being dropped or laid down in a targeted manner. It is therefore proposed, in accordance with the present disclosure, to preferably implement the stripping without additional mechanisms such as wipers or other types of active strippers by moving the electromagnet 4 further into the outer cone 11 into the third position and releasing the instruments 12 from the moving electromagnet 4 by the cone edge (see FIG. 4).

A two-stage retraction of the electromagnet 4 into a first and third position in the receptacle 11, in particular the outer cone 11, is preferably achieved pneumatically via two pressure stages of the pneumatic cylinder 6. Alternatively, a mechatronic design of the gripping device according to the present disclosure is also possible.

In the embodiment of the gripping system according to the present disclosure shown here, the implementation described below has been chosen. The upper edge of a rod 14, to which the electromagnet 4 is attached, strikes the spring base plate 15 during an upward movement while the pneumatic cylinder 6 is extending. The direction of movement is symbolized by the arrows shown in FIGS. 2, 3, and 4. In the first pressure stage and the first position shown in FIG. 3, the pneumatic cylinder 6 is operated at low pressure so that a force equilibrium is established immediately after contact with the positioning spring 16. The electromagnet 4 is thus in the centering position. The lower edge of the electromagnet 4 is not yet fully retracted into the outer cone 11 and the first position has not yet been fully reached. In the second pressure stage of the pneumatic cylinder 6, the pressure is increased and the spring 16 is further compressed so that the electromagnet 4 is fully immersed in the outer cone 11 and the first position is fully reached. The spring 16 is designed so that, at maximum force of the pneumatic cylinder 6, preferably 10 bar, it is completely compressed and, in a third position, the electromagnet 4 is retracted into the receptacle 11 (FIG. 4). All instruments 23 protruding from the underside of the electromagnet 4 are stripped off the edge of the outer cone 11.

When the cable 81 is relieved by the electromagnet 4 resting on an instrument container base 19 or an instrument 23, a further pressure spring 17 is provided in a horizontal position. This spring 17 is designed so that it is compressed by the dead weight of the electromagnet 4 and all other components, in particular the counterpart 10. When the electromagnet 4 is placed on the instrument container base 19 or an instrument 23 and the cable 81 is relieved, the spring 17 expands and thus guides the cable 81 within the precision tube 7. This prevents the cable 81 from becoming slack and jamming, for example, due to the relief in the deflection 9 or within the precision tube 7.

Basically, this design is intended to control the pneumatic cylinder 6 in two different pressure stages during the extension movement and to achieve the extension and retraction of the double-acting pneumatic cylinder 6. To ensure that the extension and retraction movements are not jerky, a throttle valve (not shown) is preferably installed in front of the inputs of the pneumatic cylinder 6. The direction of movement of the double-acting pneumatic cylinder 6 is preferably controlled by a 5/2-way valve with spring return. The electromagnetically controllable directional control valve can be connected to one of the configurable outputs of the robotic system 3 and controlled via the robot program.

The 5/2-way valve is preferably arranged and set up so that the pneumatic cylinder 6 is retracted in the position of the directional valve reset by the spring 17. As soon as the output of the robot 2 switches, the pneumatic cylinder 6 begins to extend and the electromagnet 4 is pulled into the outer cone 11 with the gripped instrument 23. To prevent the tool 23 from being stripped off directly when the electromagnet 4 is pulled too far into the outer cone 11, the extension movement of the pneumatic cylinder 6 takes place in two pressure stages.

These are preferably controlled by another 5/2-way valve. The two possible positions of the 5/2-way valve switch between two different path options with a high pressure level (preferably 2 bar) and a low pressure level (preferably 0.5 bar). A pressure reducer at the start of the first option with a low level preferably ensures a reduced pressure compared to the high level when the compressed air enters the system. In the reverse position of the directional control valve, the high pressure is applied. To prevent a brief drop in pressure due to the additional volume (of the uncommutated path), two check valves are preferably integrated at the required position. The 5/2-way valve is also controlled via a configurable output of the robotic system.

The holding device 18 of the gripping system according to the present disclosure can be designed in particular such that the electromagnet 4, preferably from the third position, can be moved to a fourth position in which the electromagnet 4 is pulled into a pull-in channel 101 of the positioning means 10. Preferably, the pneumatic cylinder 6 is provided with a further pressure stage.

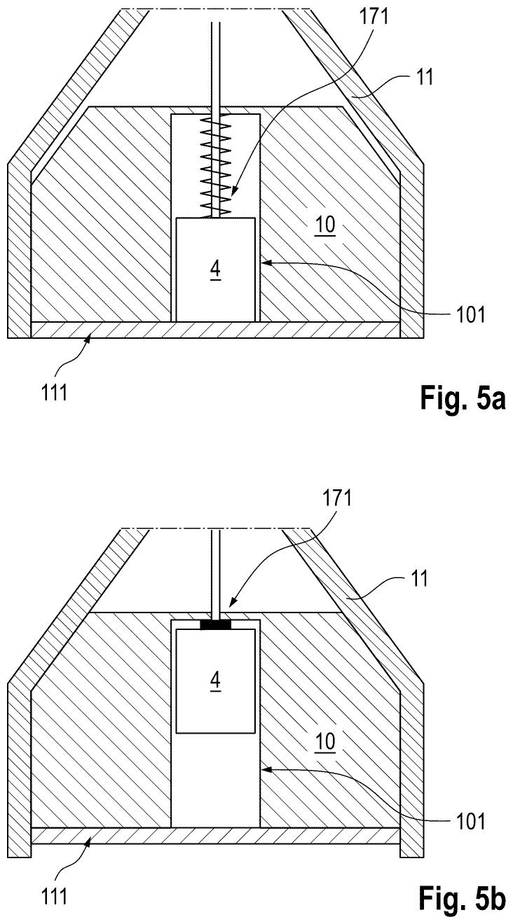

As can be seen in FIGS. 5A and 5B, a further spring 171 is arranged between the electromagnet 4 and its positioning means 10, which may be designed as a lowerable housing 10, around the cable 81. This spring 171 preferably has a higher spring constant than the spring 16 of the positioning spring 16. The spring 171 is arranged between the magnet 4 and the upper end of the positioning means 10, in particular the upper end of the pull-in channel 101 of the positioning means 10. In addition, the feed channel 101 is closed at its end intended for receiving instruments with a cover 111, which is complete here. The positioning means 10 can be manufactured in one piece with the cover 111 as an integral component or in several parts, e.g., as a cap.

The further pressure stage in the fourth position is used if the stripping of instruments 23 in the third position was not successful. This can happen if an instrument 23 is hanging from the electromagnet 4 with the instrument syringe (i.e., virtually vertically), as shown in FIG. 4, and therefore does not come into contact with the outer cone 11 when the electromagnet 4 moves into the outer cone 11. If the fourth position shown in FIG. 5b is now approached from the third position also shown in FIG. 5a and the pressure is increased relative to the third position, the electromagnet 4 moves upward from the position shown in FIG. 5a in the feed channel 101 of the positioning means 10 (see FIG. 5b) and compresses the further spring 171. In doing so, the electromagnet 4 moves away from the residual magnetized instrument, which is separated from the electromagnet 4 by the cover 111, whereby the latter is deposited. The positioning means 10 comes into contact with the receptacle 11, preferably in the area of the outer cone 11, either when the third pressure stage is reached or when the third pressure stage transitions to the fourth pressure stage.

### LIST OF REFERENCE SIGNS

1 Gripping device
2 Adapter device, robot interface
3 Robotic system, in particular 6 DOF articulated arm robot
4 Electromagnet
5 Adapter plate
6 Pneumatic cylinder
7 Precision tube
8 Steering means
81 Cable, preferably steel cable,
9 Outer sleeve, deflection
10 Positioning means, counterpart
101 Pullin channel
11 Receptable, outer cone
12 Adapter
13 Sliding bearing
14 Rod
15 Spring base plate
16 Positioning spring
17 Compression spring
18 Holding device
19 Working surface
20 Drive unit
21 Optical system
22 Control unit
23 Instrument
24 Instrument container
171 Additional spring

1. A gripping system for gripping and positioning medical instruments, the gripping system comprising:
a) a gripping device comprising an electromagnet and configured for receiving magnetizable instruments from an instrument container via the electromagnet;
b) an adapter device configured for attaching the gripping device to a control system; and
c) a holding device that attaches the electromagnet to the adapter device,
the holding device comprising a receptacle for receiving the electromagnet, and
the holding device being arranged such that the electromagnet is movable between a first position, in which the electromagnet is received in the receptacle, and a second position, in which the electromagnet is extended out of the receptacle.

2. The gripping system according to claim 1, wherein the control system is a robotic system.

3. The gripping system according to claim 1, wherein the holding device comprises a cone.

4. The gripping system according to claim 1, wherein the holding device comprises a steering element configured to move the electromagnet transversely to a working surface.

5. The gripping system according to claim 4, wherein the steering element comprises at least one flexible section.

6. The gripping system according to claim 4, wherein the holding device comprises a drive unit configured to move the electromagnet and/or the steering element.

7. The gripping system according to claim 6, wherein the drive unit is a pneumatic unit.

8. The gripping system according to claim 7, wherein the pneumatic unit comprises a pneumatic cylinder configured to move the electromagnet and/or the steering element by at least two different pressure stages of the pneumatic cylinder.

9. The gripping system according to claim 6, further comprising a control unit configured to transmit control commands to the drive unit for moving the electromagnet.

10. The gripping system according to claim 9, wherein the control unit is configured to detect instruments in an empty instrument container.

11. The gripping system according to claim 10, wherein the control unit is configured to detect instruments in an empty instrument container using image data detected by an optical system.

12. The gripping system according to claim 6, wherein the electromagnet is mounted on the steering element and freely oscillates when in the second position.

13. The gripping system according to claim 1, wherein the holding device comprises at least one compression spring, and wherein the electromagnet has a dead weight configured to compress the at least one compression spring.

14. The gripping system according to claim 1, wherein the holding device comprises at least one positioning spring configured to position the electromagnet in the receptacle.

15. The gripping system according to claim 1, wherein the holding device is arranged such that the electromagnet is movable from the first position and/or the second position into a third position in which the electromagnet is retracted into the receptacle.

16. The gripping system according to claim 15, further comprising a housing into which the electromagnet is received.

17. The gripping system according to claim 16, wherein the holding device is arranged such that the electromagnet is movable from the third position, into a fourth position in which the electromagnet is drawn into a pull-in channel of the housing.

18. A method for removing surgical instruments from an instrument container using the gripping system according to claim 1, the method comprising the steps of:
activating the electromagnet;
moving the electromagnet from the first position to the second position;
picking up a magnetizable instrument with the electromagnet in the second position;
moving the electromagnet from the second position to the first position; and
deactivating the electromagnet.

19. The method according to claim 18, further comprising the step of retracting the electromagnet from the first position and/or the second position to a third position in which the electromagnet is retracted into the receptacle, thereby stripping off the magnetizable instrument.

20. The method according to claim 19, further comprising the step of pulling the electromagnet from the third position into a fourth position, in which the electromagnet is pulled into a pull-in channel of a housing.
